(19) United States
(12) Patent Application Publication
(10) Pub. No.: US 2025/0205501 A1
(43) Pub. Date: Jun. 26, 2025

(54) SYSTEM AND METHOD FOR IMPLANT DETECTION AND ELECTROMAGNETIC FIELD DIAGNOSIS

(71) Applicant: Cochlear Limited, Macquarie University, NSW (AU)

(72) Inventors: Kostas Ioannis Tsampazis, North Ryde (AU); Shaun Ashwin Kumar, Arncliffe (AU)

(21) Appl. No.: 18/848,679

(22) PCT Filed: Mar. 16, 2023
(86) PCT No.: PCT/IB2023/052593

§ 371 Date: Sep. 19, 2024

Related U.S. Application Data
(60) Provisional application No. 63/380,871, filed on Oct. 25, 2022.
(60) Provisional application No. 63/363,530, filed on Apr. 25, 2022.

(51) Int. Cl. A61N 1/372; A61N 1/36; A61N 1/378

(52) U.S. Cl. CPC A61N 1/37229; A61N 1/36038; A61N 1/37276; A61N 1/3787

(57) ABSTRACT

An apparatus includes at least one external antenna external to a recipient's body and configured to be in wireless communication with at least one internal antenna of an implanted device within the recipient's body. The apparatus further includes at least one electromagnetic field detector external to the recipient's body and configured to generate detector signals in response to electromagnetic fields generated by the at least one external antenna, by the at least one internal antenna. and/or by other electromagnetic field sources. The apparatus further includes circuitry in operable communication with the at least one external antenna and the at least one electromagnetic field detector. The circuitry is configured to receive the detector signals from the at least one electromagnetic field detector and. in response at least in part to the detector signals. transmit control signals to the at least one external antenna. A method can use the detector signals to detect an operational state of the apparatus and/or to automatically optimize power and/or data transmission between the apparatus and the implanted device.

### BACKGROUND

### Field

The present application relates generally to systems and methods for facilitating wireless power and/or information transmission from a first device to a second device, and more specifically, for facilitating wireless power and/or information transmission from an external portion of a medical system to an implanted portion of the medical system.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises at least one external antenna external to a recipient's body and configured to be in wireless communication with at least one internal antenna of an implanted device within the recipient's body. The apparatus further comprises at least one electromagnetic field detector external to the recipient's body and configured to generate detector signals in response to electromagnetic fields generated by the at least one external antenna, by the at least one internal antenna, and/or by other electromagnetic field sources. The apparatus further comprises circuitry in operable communication with the at least one external antenna and the at least one electromagnetic field detector. The circuitry is configured to receive the detector signals from the at least one electromagnetic field detector and, in response at least in part to the detector signals, transmit control signals to the at least one external antenna.

In another aspect disclosed herein, a method comprises wirelessly transmitting electrical power and/or data from an external portion of a medical implant system outside a recipient's body. The external portion is configured to be in wireless communication with an internal portion of the medical implant system within the recipient's body. The method further comprises detecting electromagnetic fields generated by the external portion, by the internal portion, and/or by other electromagnetic field sources. The method further comprises generating information in response to the detected electromagnetic fields. The information is indicative of at least one of: a presence or absence of the internal portion; a distance between the external portion and the internal portion; a presence or absence of external interference of wireless communication between the external portion and the internal portion; an operational state of the external portion of the medical implant; and a coupling strength between the external portion and the internal portion.

In another aspect disclosed herein, a method comprises wirelessly transmitting electrical power and/or data from an external portion of a medical implant system outside a recipient's body. The method further comprises detecting electromagnetic fields generated by the external portion. The method further comprises generating information in response to the detected electromagnetic fields. The information is indicative of a presence or absence of an internal portion of the medical implant system implanted within the recipient's body. The method further comprises, in response to the information being indicative of an absence of the internal portion, terminating said wirelessly transmitting electrical power and/or data from the external portion.

In another aspect disclosed herein, a method comprises wirelessly transmitting electrical power and/or data from an external portion of a medical implant system outside a recipient's body. The method further comprises detecting electromagnetic fields generated by the external portion. The method further comprises generating information in response to the detected electromagnetic fields, the information indicative of an operational state of the external portion. The method further comprises, in response to the information, modifying said wirelessly transmitting electrical power and/or data from the external portion.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIGS. 1B-1D schematically illustrate other example cochlear implant auditory prostheses in accordance with certain implementations described herein;

FIG. 2A schematically illustrates an example apparatus in accordance with certain implementations described herein;

FIG. 2B schematically illustrates the example apparatus of FIG. 2A comprising an external portion of an acoustic prosthesis system in accordance with certain implementations described herein;

FIG. 3A is a flow diagram of an example method in accordance with certain implementations described herein;

FIG. 3B is a flow diagram of another example method in accordance with certain implementations described herein;

FIG. 3C is a flow diagram of still another example method in accordance with certain implementations described herein;

FIGS. 4A-4E schematically illustrate plots of the detected electromagnetic fields as functions of time in accordance with certain implementations described herein;

FIGS. 5A and 5B plot the detected electromagnetic fields during a power transmission phase using first wire coil having a tuning frequency of 5 MHz for two different SFT values in accordance with certain implementations described herein;

FIG. 6 plots the electric voltage corresponding to electromagnetic fields detected during a quiet phase in accordance with certain implementations described herein; and

FIGS. 7A-7C are a flow diagram of an example method of detection of an internal device and diagnosis of the RF communication link between an external device and the internal device in accordance with certain implementations described herein.

### DETAILED DESCRIPTION

In certain implementations disclosed herein, a system and method is configured to measure electromagnetic fields between the antennas of an external device outside a recipient's body and an internal device implanted within the recipient's body and having a wireless RF link with the external device (e.g., between an externally worn sound processor and an implanted cochlear implant) and/or electromagnetic fields generated by the external device. The external device can comprise a detector (e.g., a wire coil and differential amplifier having a large impedance) configured to generate signals (e.g., electric voltages) indicative of the measured electromagnetic fields without appreciably affecting the electromagnetic fields. The detector can serve as a feedback sensor adding diagnostic capabilities to the external device in which the operation of the external device can be evaluated and/or various internal and external parameters that can affect the RF link are detected and measured in real time. As a result, an adaptive and more robust RF link can be achieved (e.g., minimum iterations and maximum robustness).

The detection signals generated during power transmission bursts, data transmission bursts, and/or periods during which no transmission is attempted, can be used for one or more of: detecting the presence or absence of the internal device, measuring the skin flap thickness and/or coupling strength between the external and internal devices, detecting and measuring external interference, adjusting the transmission electromagnetic field strength to automatically optimize power and/or data transmission in view of the coupling strength and/or external interference, and to extract or restore telemetry pulses. The detection signals can also be used to detect when conditions are such that coupling between the external device and the internal device are degraded and to either automatically apply a solution (e.g., automatically increase the transmission electromagnetic field strength; automatically cease transmission when no internal device is coupled, thereby conserving power) and/or to notify the recipient and/or a practitioner to apply a solution (e.g., move out of the region where excessive external interference is detected; reposition the external device relative to the implanted device).

The teachings detailed herein are applicable, in at least some implementations, to any type of implantable medical device (e.g., implantable sensory prostheses) comprising a first portion (e.g., external to a recipient) and a second portion (e.g., implanted on or within the recipient), the first portion configured to wirelessly transmit power and/or information (e.g., data; commands) to the second portion. For example, the implantable medical device can comprise an auditory prosthesis system utilizing an external sound processor configured to transcutaneously provide power and/or information to an implanted assembly (e.g., comprising an actuator). In certain such examples in which the external sound processor is configured to transcutaneously provide information (e.g., data signals; control signals) to the implanted assembly, the implanted assembly is configured to respond to the information by generating stimulation signals that are perceived by the recipient as sounds. In addition, the external sound processor can be configured to transcutaneously receive information (e.g., data signals; control signals) from the implanted assembly. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other medical devices that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; etc. The concepts described herein and/or variations thereof can be applied to any of a variety of implantable medical devices comprising an implanted component configured to use magnetic induction to receive power and/or information (e.g., transcutaneously) from an external component and to store at least a portion of the power in at least one power storage device (e.g., battery; tank capacitor). The implanted component can also be configured to receive control signals from the external component (e.g., transcutaneously) and/or to transmit sensor signals to the external component (e.g., transcutaneously) while receiving power from the external component. In still other implementations, the teachings detailed herein and/or variations thereof can be utilized in other types of systems beyond medical devices utilizing magnetic induction for wireless power transfer. For example, such other systems can include one or more of the following: consumer products (e.g., smartphones; “internet-of-things” or IoT devices) and electric vehicles (e.g., automobiles).

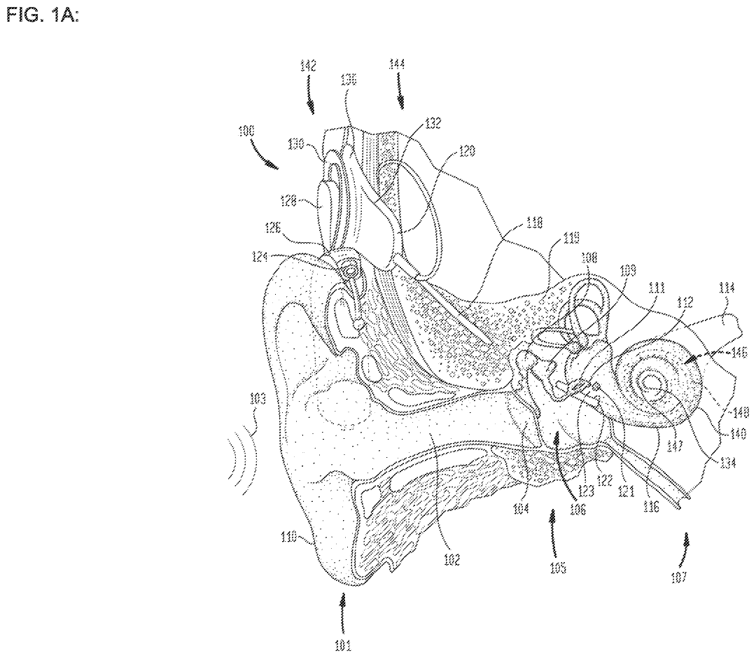

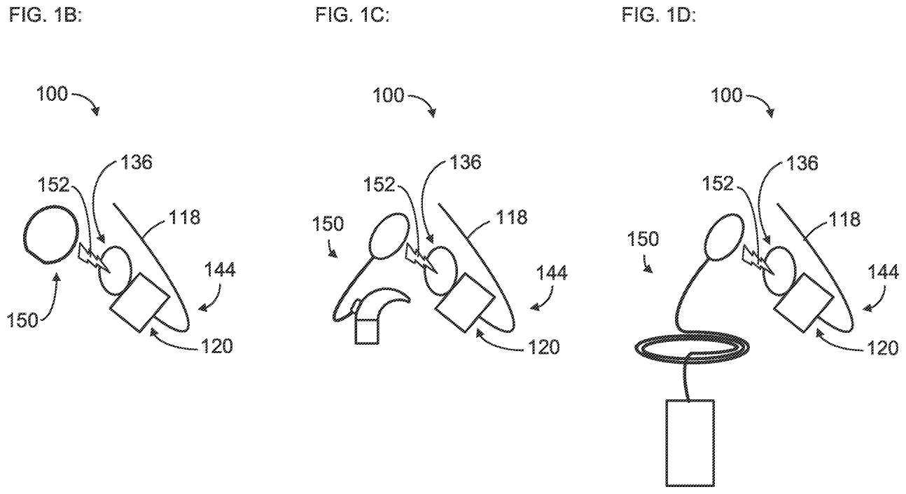

FIGS. 1A-1D show various example systems 100 compatible with certain implementations described herein. FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1A with a subcutaneously implantable assembly comprising an acoustic transducer (e.g., microphone), as described more fully herein.

As shown in FIG. 1A, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1A) (e.g., audio ports, such as a Direct Audio Input (DAI); data ports, such as a Universal Serial Bus (USB) port; cable ports, etc.). In the example of FIG. 1A, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1A) (e.g., battery; tank capacitor), a processing module (not shown in FIG. 1A) (e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1A, the external transmitter unit 128 comprises circuitry that includes at least one external inductive coil 130 (e.g., a wire antenna coil comprising multiple turns of electrically insulated copper wire). The external transmitter unit 128 also generally comprises a magnet (not shown in FIG. 1A) secured directly or indirectly to the at least one external inductive coil 130. The at least one external inductive coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. IA, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive coil 136 (e.g., a wire antenna coil comprising multiple turns of electrically insulated single-strand or multi-strand platinum or gold wire), and generally, a magnet (not shown in FIG. 1A) fixed relative to the at least one internal inductive coil 136. The at least one internal inductive coil 136 receives power and/or data signals from the at least one external inductive coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIGS. 1B-1D schematically illustrate other example cochlear implant auditory prosthesis systems 100 in accordance with certain implementations described herein. The example auditory prosthesis systems 100 of FIGS. 1B-1D comprise an internal component 144 comprising an internal inductive coil 136, an internal microphone (not shown), and a stimulation unit 120 comprising an elongate stimulation assembly 118. The example auditory prosthesis systems 100 of FIGS. 1B-1D further comprise an external device 150 configured to wirelessly transfer power and/or information signals 152 to the internal component 144 (e.g., to charge the internal component 144). In certain implementations, the external device 150 can be further configured to receive load modulation backlink data from the internal component 144 during the wireless power transfer. In FIG. 1B, the external device 150 comprises an “off-the-ear” (OTE) sound processing and charging device comprising sound processing circuitry and power transmitting circuitry (e.g., coil) within the same housing. In FIG. 1C, the external device 150 comprises a “behind-the-ear” (BTE) sound processing and charging device comprising sound processing circuitry and power transmitting circuitry (e.g., coil) within separate housings having a wired connection therebetween. In FIG. 1D, the external device 150 comprises a dedicated charging device (e.g., the external device 150 does not provide stimulation data to the internal component 144). The external device 150 comprises power transmitting circuitry (e.g., coil) and a power source (e.g., configured to be worn on the recipient's body) within separate housings having a wired connection therebetween. In certain other implementations, the external device 150 comprises a wireless power transmission (WPT) charger (e.g., pillow charger) configured to wirelessly transfer power to the internal component 144 upon the recipient laying the body portion containing the internal component 144 on or within an operative region of the WPT charger.

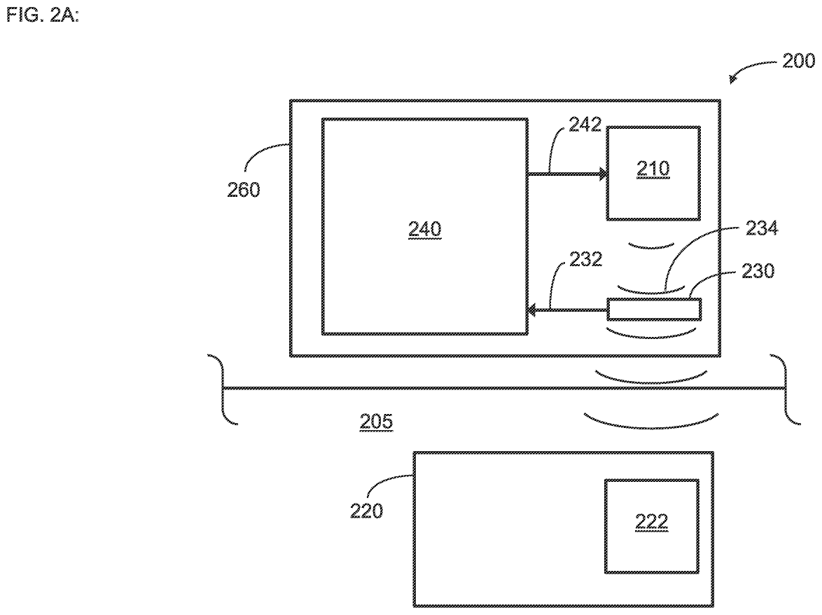

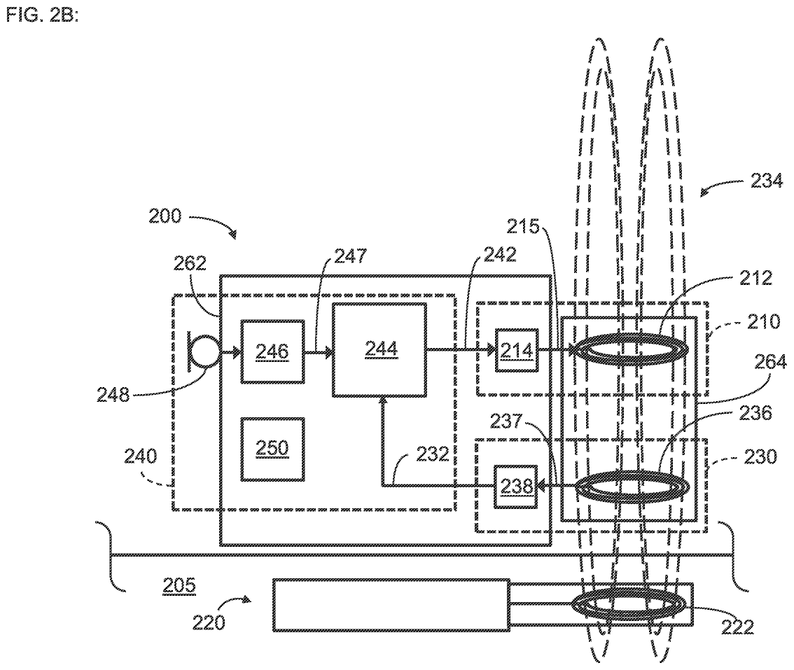

FIG. 2A schematically illustrates an example apparatus 200 in accordance with certain implementations described herein. The apparatus 200 comprises at least one external antenna 210 external to a recipient's body 205 and configured to be in wireless communication with at least one internal antenna 222 of an implanted device 220 within the recipient's body 205. The apparatus 200 further comprises at least one electromagnetic field detector 230 external to the recipient's body 205 and configured to generate detector signals 232 in response to electromagnetic fields 234 generated by the at least one external antenna 210, by the at least one internal antenna 222, and/or by other electromagnetic field sources. The apparatus 200 further comprises circuitry 240 in operable communication with the at least one external antenna 210 and the at least one electromagnetic field detector 230. The circuitry 240 is configured to receive the detector signals 232 from the at least one electromagnetic field detector 230 and, in response at least in part to the detector signals 232, transmit control signals 242 to the at least one external antenna 210.

FIG. 2B schematically illustrates the example apparatus 200 of FIG. 2A comprising an external portion of an acoustic prosthesis system (e.g., a portion worn by the recipient; a portion that is configured to be repeatedly attached to and detached from the recipient; an external device 150 of a cochlear implant auditory prosthesis system 100; see, e.g., FIGS. 1A-1D) in accordance with certain implementations described herein. The apparatus 200 is configured to wirelessly transmit power and/or data to an internal portion of the acoustic prosthesis system (e.g., a portion implanted on or within the recipient; internal component 144 of the cochlear implant auditory prosthesis system 100). In certain implementations, the apparatus 200 comprises at least one magnet (not shown) configured to interact with a magnetic material of the internal device 220 to create an attractive magnetic force that adheres the apparatus 200 to the recipient's body in an operative position relative to the internal device 220.

In certain implementations, the at least one external antenna 210 is configured to be inductively coupled to the at least one internal antenna 222 of the implanted device 220 such that the at least one external antenna 210 can transmit power and/or information (e.g., signals; data) to the implanted device 220 and/or can receive information (e.g., signals; data) from the implanted device 220. As shown in FIG. 2B, the circuitry 240 can comprise processor circuitry 244, microphone interface circuitry 246 in operative communication with at least one microphone 248, and a power source 250 (e.g., at least one battery; at least one capacitor) configured to provide power to the processor circuitry 244. For example, the processor circuitry 244 can comprise digital signal processing circuitry (e.g., one or more digital signal processors (DSPs), one or more microcontroller cores, one or more application-specific integrated circuits (ASICs)) configured to receive data signals 247 from the microphone interface circuitry 246, the data signals 247 indicative of sound received by the at least one microphone 248. The processor circuitry 244 can be configured to generate the control signals 242 in response, at least in part, to the detector signals 232 and the data signals 247.

In certain implementations, the at least one external antenna 210 comprises a first wire coil 212 (e.g., a substantially planar inductive coil) and at least one antenna driver 214 in operative communication with the first wire coil 212. The apparatus 200 of certain such implementations is configured to be worn on the recipient's body 205 such that the first wire coil 212 is substantially parallel to a substantially planar wire coil of the at least one internal antenna 222. The at least one antenna driver 214 can be configured to provide electrical current 215 to the first wire coil 212 in response to the control signals 242, such that the first wire coil 212 generates the electromagnetic field 234 (e.g., magnetic field) to wirelessly communicate with the implanted device 220.

In certain implementations, the at least one electromagnetic field detector 230 comprises a second wire coil 236 (e.g., a substantially planar inductive coil) and a detector interface 238 in operative communication with the second wire coil 236. The second wire coil 236 can be positioned to intercept at least a portion of the magnetic flux generated by the first wire coil 212 (e.g., the second wire coil 236 bounding an area through which the portion of the magnetic flux flows). For example, as schematically illustrated by FIG. 2B, the second wire coil 236 can be positioned between the at least one external antenna 210 and the recipient's body 205 (e.g., with the first wire coil 212 and the second wire coil 236 both substantially planar and substantially parallel to one another). In certain implementations, the second wire coil 236 can have a size (e.g., radius; width) that is the same or smaller than that of the first wire coil 212 and/or a number of coil turns (e.g., 2, 3, or more) that is the same or smaller than that of the first wire coil 212.

In response to the intercepted portion of the magnetic flux, the second wire coil 236 can generate an electrical voltage 237 received by the detector interface 238. The detector interface 238 can comprise a differential amplifier having sufficiently large impedance that an electrical current does not flow within the second wire coil 236 in response to the electromagnetic fields 234 (e.g., the second wire coil 236 is not loaded; the second wire coil 236 is used in open circuit mode). In this way, the at least one electromagnetic field detector 230 can avoid substantially affecting the electromagnetic fields 234 (e.g., operates as a passive probe that does not affect the performance of the external and internal antennas 210, 222). The gain of the differential amplifier can be in a range of 1 to 10 and can be selected such that a signal-to-noise ratio of the detector signals 232 is sufficient for operation by the apparatus 200.

In certain implementations, the apparatus 200 comprises at least one housing 260 containing the at least one external antenna 210, the at least one electromagnetic field detector 230, and the circuitry 240. For example, the at least one housing 260 can comprise a single housing or the at least one housing 260 can comprise multiple housings containing different portions of the apparatus 200. As schematically illustrated by FIG. 2B, the apparatus 200 comprises a first housing 262 containing at least a portion of the circuitry 240 (e.g., the processor circuitry 244, the microphone interface circuitry 246, the power source 250), a portion of the at least one external antenna 210 (e.g., the at least one antenna driver 214), and a portion of the at least one electromagnetic field detector 230 (e.g., the detector interface 238), and a second housing 264 containing the other portion of the at least one external antenna 210 (e.g., the first wire coil 212) and the other portion of the at least one electromagnetic field detector 230 (e.g., the second wire coil 236). The at least one microphone 248 can be on or within the first housing 262 or spaced from the first housing 262.

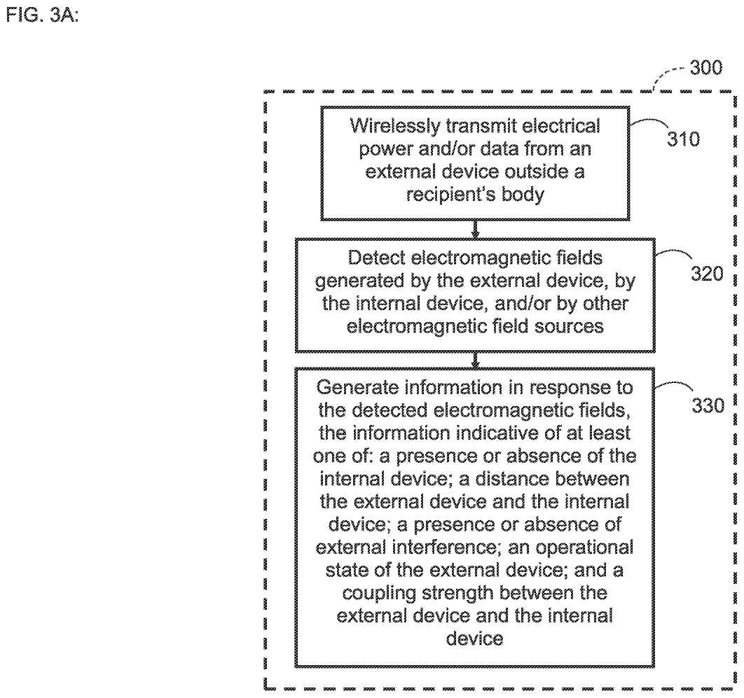

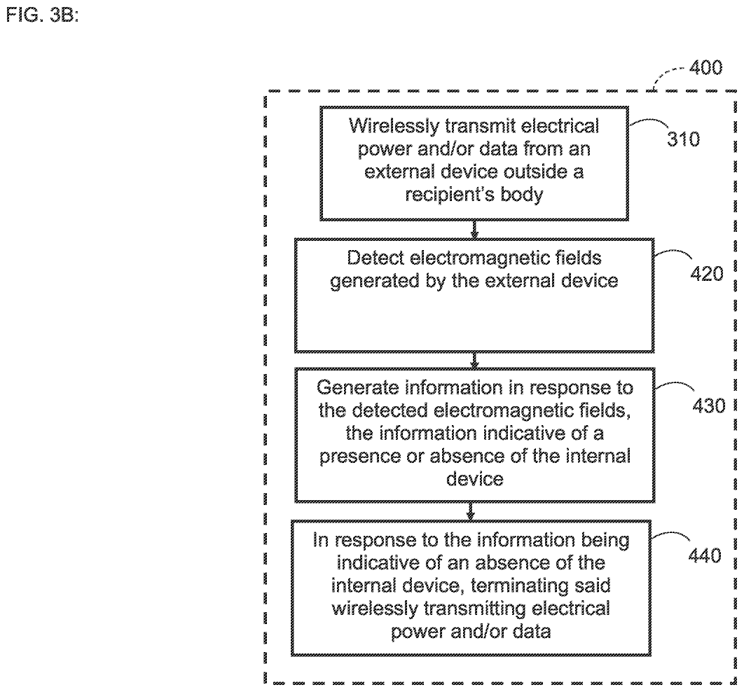

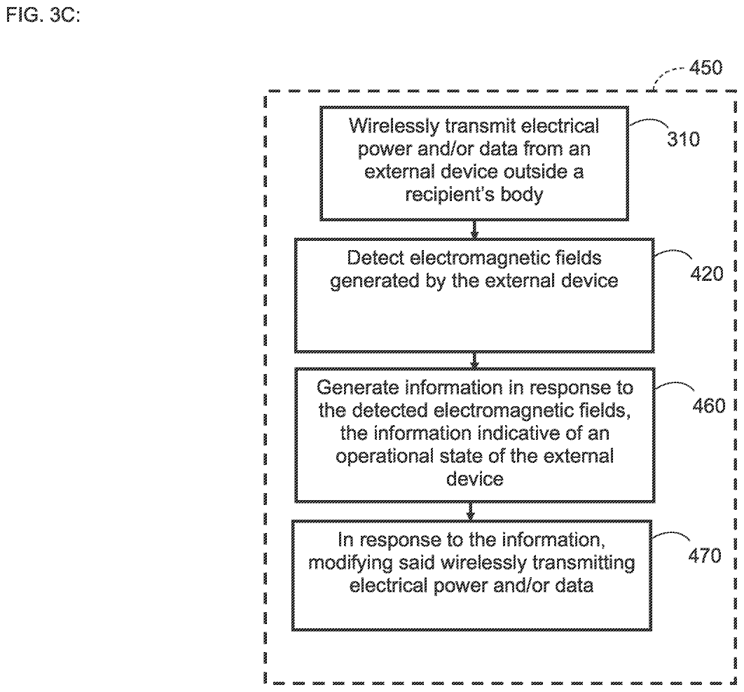

FIG. 3A is a flow diagram of an example method in accordance with certain implementations described herein. FIG. 3B is a flow diagram of another example method 400 in accordance with certain implementations described herein. FIG. 3C is a flow diagram of still another example method 450 in accordance with certain implementations described herein. The example methods 400, 450 are specific examples of the method 300 which, respectively, further address the circumstance of an absence of the implanted device 220 and an aberrant operational state and/or a malfunction of the example apparatus 200. While the methods 300, 400, 450 are described by referring to some of the structures of the example apparatus 200 of FIGS. 1A-1D and 2A-2B, other apparatus and systems with other configurations of components can also be used to perform the methods 300, 400, 450 in accordance with certain implementations described herein. For example, the methods 300, 400, 450 can be performed by an external portion of a medical device or system (e.g., an auditory or visual prosthesis system; cardiac pacemaker or defibrillator system) configured to have a magnetic induction link configured to transmit power and/or data to an internal portion (e.g., implanted component) of the medical device or system.

In an operational block 310, the method 300 comprises wirelessly transmitting electrical power and/or data from an external device (e.g., the apparatus 200; an external portion of a medical implant system, such as an auditory prosthesis system) outside a recipient's body. The external device is configured to be in wireless communication with an internal device (e.g., the implanted device 220; an internal or implanted portion of the medical implant system) within the recipient's body (e.g., to wirelessly transmit the power and/or data through at least a portion of the recipient's body to the internal device). Each of the method 400 and the method 450 also comprises the operational block 310. In certain implementations of the method 300 in which the external device is in wireless communication with the internal device, at least a portion of the electrical power and/or data transmitted from the external device is received by the internal device. In certain other implementations of the method 300 in which the external device is not in operable communication with the internal device, the electrical power and/or data transmitted from the external device is not received by another device (e.g., the internal device).

In an operational block 320, the method 300 further comprises detecting electromagnetic fields 234 generated by the external device, by the internal device, and/or by other electromagnetic field sources. For example, the at least one electromagnetic field detector 230 can receive at least a portion of the electromagnetic fields 234 (e.g., intercept magnetic flux) and can generate and transmit the detector signals 232 to the circuitry 240. Each of the method 400 and the method 450 comprises an operational block 420 similar to operational block 320, in which electromagnetic fields 234 generated by the external device are detected.

In an operational block 330, the method 300 further comprises generating information in response to the detected electromagnetic fields 234. For example, based at least in part on the detector signals 232 received from the at least one electromagnetic field detector 230, the circuitry 240 can generate the information. In certain implementations, the information is indicative of at least one of: a presence or absence of the internal device; a distance between the external device and the internal device; a presence or absence of external interference of wireless communication between the external device and the internal device; an operational state of the external device; and a coupling strength between the external device and the internal device. The method 400 comprises an operational block 430 similar to operational block 330, in which the generated information is indicative of the presence or absence of the internal device. The method 450 comprises an operational block 460 similar to operational block 330, in which the generated information is indicative of the operational state of the external device.

In an operational block 440, the method 400 comprises, in response to the information being indicative of an absence of the internal device, terminating said wirelessly transmitting electrical power and/or data from the external device. In certain implementations, the method 400 further comprises, in response to the information being indicative of an absence of the internal device, generating a signal indicative of the absence of the internal device, the signal configured to be received by a user of the medical implant system (e.g., the recipient; a practitioner diagnosing the medical implant system). For example, this signal can be presented via a display device or an indicator light (LED). In certain implementations, the information is generated in response, at least in part, to the detected electromagnetic fields 234 while wirelessly transmitting electrical power from the apparatus 200 (e.g., during a power transmission phase of the apparatus 200).

In an operational block 470, the method 450 comprises, in response to the information, modifying said wirelessly transmitting electrical power and/or data from the external device. For example, the method 450 can comprise identifying, in response to the information, an aberrant operational state and/or a malfunction of the external device. In certain implementations, the method 450 further comprises, in response to the information, generating a signal indicative of the operational state and/or the malfunction of the external device, the signal configured to be received by a user of the medical implant system (e.g., the recipient; a practitioner diagnosing the medical implant system). For example, this signal can be presented via a display device or an indicator light (LED). In certain implementations, the information is generated in response, at least in part, to the electromagnetic fields 234 detected while the external device is not in operational communication with the internal device.

In certain implementations, said detecting the electromagnetic fields comprises measuring a duration and amplitude of RF fields generated by the external device during a power transmission operational phase (e.g., power burst) and/or during a data transmission operational phase (e.g., data burst). This measurement can confirm proper operation of the external device or can detect and/or identify malfunctions of the external device prior to the external device being placed in operational communication with the internal device. In certain such implementations, the measurements can be part of a full self-diagnostic operation on the functionality of the external device (e.g., to diagnose problems with the external device before they become worse).

A first type of malfunction (e.g., faulty operational condition) can be detected when the measured duration of the power transmission operational phase is greater than a predetermined (e.g., previously stored in memory) threshold value (e.g., only a continuous RF power supply signal being transmitted with no data bursts being transmitted) or a measured duration of a time period between sequential power transmission operational phases is less than a predetermined threshold value. In the first type of malfunction, the data communication between the external device and the internal device can be affected (e.g., a data transmission phase and/or telemetry pulses can be corrupted and/or nonexistent) and can be caused by corruption of the firmware of the external device. In addition, the first type of malfunction can cause excessive energy and/or power transferred to the internal device, resulting in overheating of the internal device and/or possible nerve/tissue damage to the recipient. If the external device detects the first type of malfunction, then the controller of the external device can adjust (e.g., terminate) the power transmission operational phase (e.g., to prevent overheating of the internal device) and/or can inform the user (e.g., recipient and/or practitioner). For example, the signal can prompt the user to initiate an update of the firmware of the external device and/or to avoid placing the external device in operational communication with the internal device thereby preventing further damage to the external device, the internal device, and/or the recipient.

A second type of malfunction can be detected when the measured amplitude of the RF power supply signal during the power transmission operational phase (e.g., power burst) is below a predetermined threshold value (e.g., low measured amplitude caused by faulty operation of the circuitry 240 and/or of a battery of the external device) such that upon the external device being in operational communication with the internal device, the internal device would be unable to perform normal operations. The second type of malfunction can be caused by current leakages that can result in overheating and/or damage to the external device. If the external device detects the second type of malfunction, then the controller of the external device can inform the user (e.g., recipient and/or practitioner) so that the user can avoid placing the external device in operational communication with the internal device, thereby preventing further damage to the external device and/or the recipient. In certain implementations, besides informing the user, the controller of the external device can assess (e.g., evaluate) whether the low measured amplitude is still sufficient for proper functionality of the system or whether the low measured amplitude is insufficient for proper functionality of the system (e.g., in response to which the controller can attempt corrective actions, such as adjusting or recalculating the coupling coefficient between the communication coils).

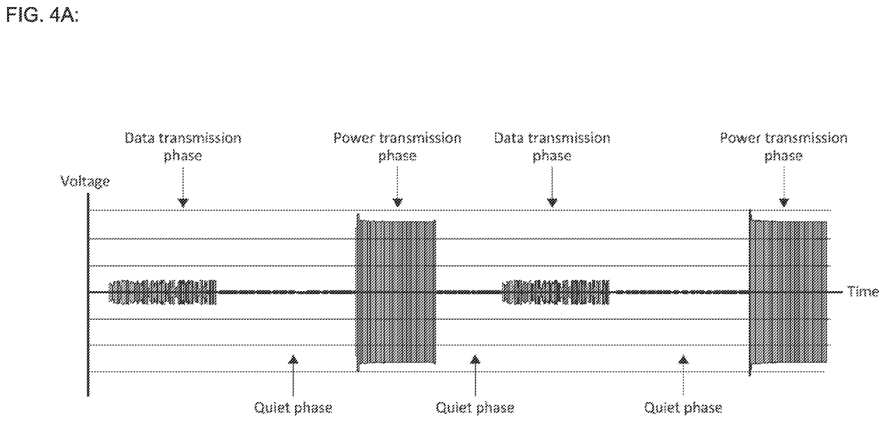

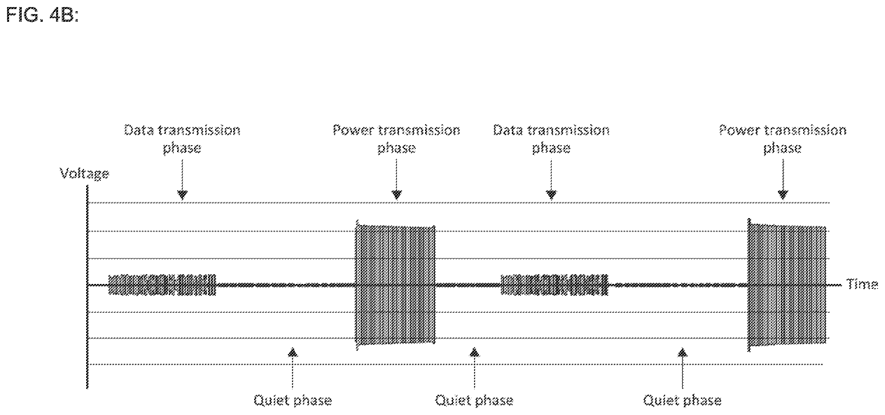

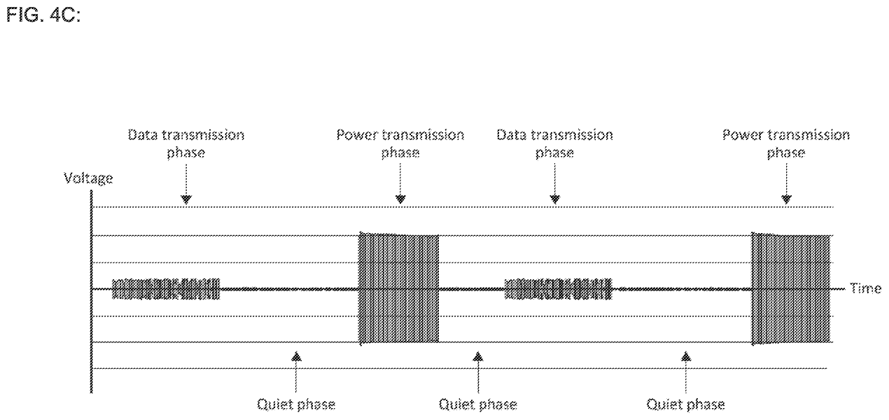

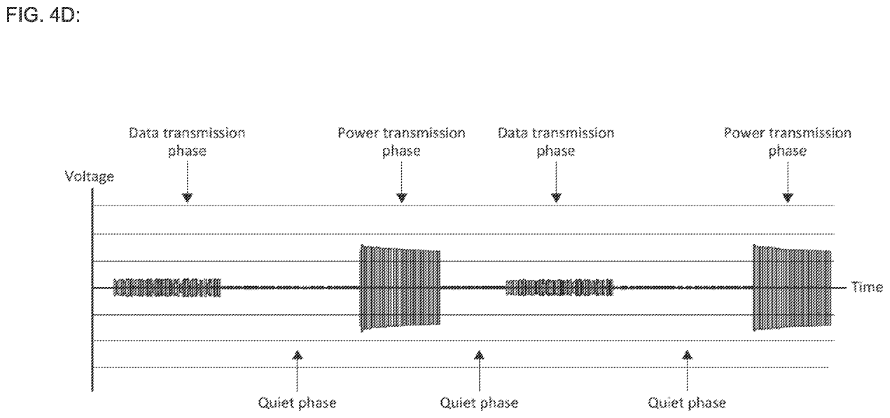

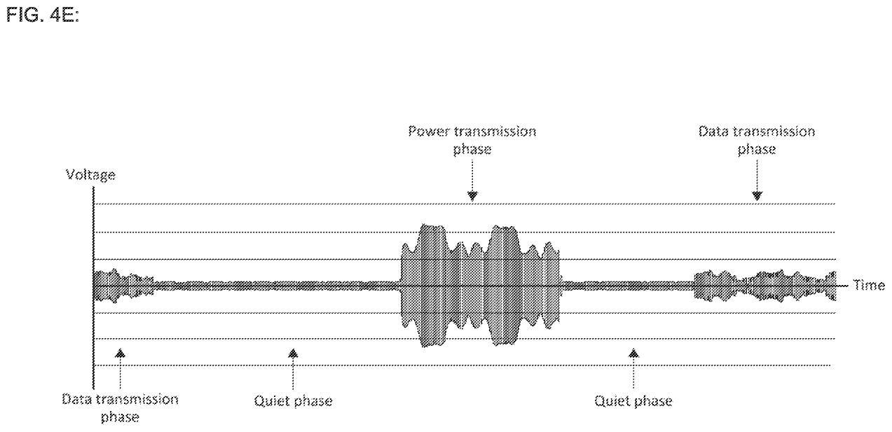

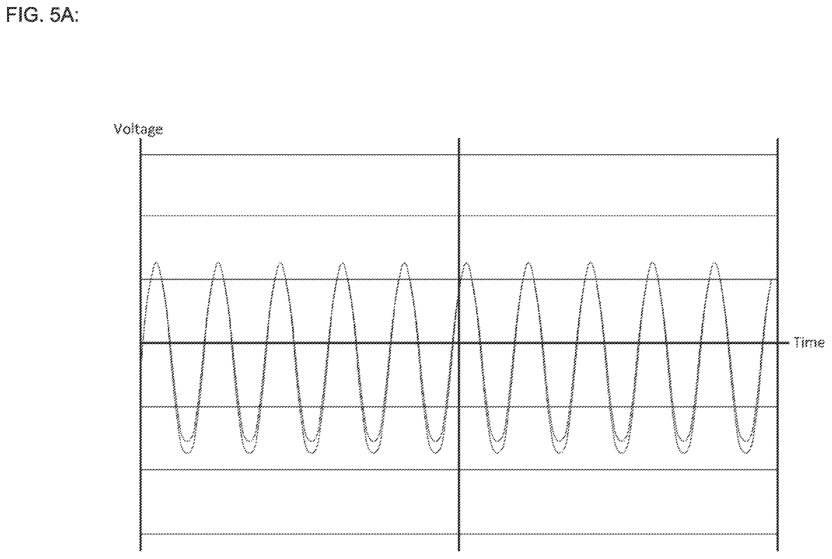

FIGS. 4A-4E schematically illustrate plots of the detected electromagnetic fields 234 (e.g., the electric voltage 237 induced into the second wire coil 236 of the at least one electromagnetic field detector 230) as functions of time in accordance with certain implementations described herein. The electromagnetic fields 234 can be detected while wirelessly transmitting electrical power from the external device (e.g., during a power transmission phase of the apparatus 200), while wirelessly transmitting data from the external device (e.g., during a data transmission phase of the apparatus 200), and/or while not wirelessly transmitting electrical power or data from the external device (e.g., during a “quiet” phase of the apparatus 200). Each of FIGS. 4A-4E shows a series of data transmission phases, quiet phases, and power transmission phases. The electrical current flowing through the first wire coil 212 of the at least one external antenna 210 during the power transmission phases of FIGS. 4A-4E are the same as one another, and the electrical current flowing through the first wire coil 212 of the at least one external antenna 210 during the data transmission phases of FIGS. 4A-4E are the same as one another and less than the electrical current during the power transmission phases. There is no electrical current flowing through the first wire coil 212 of the at least one external antenna 210 during the quiet phases of FIGS. 4A-4E.

FIG. 4A shows the electromagnetic fields 234 detected when the apparatus 200 is not in wireless communication with the implanted device 220. For example, the apparatus 200 can be positioned such that the first wire coil 212 of the at least one external antenna 210 is not operationally coupled with at least one internal antenna 222 of an implanted device 220 (e.g., there is no implanted device 220 beneath the portion of the recipient's skin on which the apparatus 200 is positioned or the first wire coil 212 is too far from the at least one internal antenna 222 of an implanted device 220). FIGS. 4B-4D show the detected electromagnetic fields 234 for different skin flap thicknesses (SFT) of the recipient's tissue between the at least one external antenna 210 of the apparatus 200 and the at least one internal antenna 222 of the implanted device 220 (e.g., SFT can vary among different recipients in a range of 1 millimeter to 15 millimeters). FIG. 4B plots the detected electromagnetic fields 234 when the first wire coil 212 is over the internal antenna 222 with a distance (e.g., SFT) of about 10 millimeters from the internal antenna 222. FIG. 4C plots the detected electromagnetic fields 234 when the first wire coil 212 is over the internal antenna 222 with a distance (e.g., SFT) of about 5 millimeters from the internal antenna 222. FIG. 4D plots the detected electromagnetic fields 234 when the first wire coil 212 is over the internal antenna 222 with a distance (e.g., SFT) of about 1 millimeter from the internal antenna 222. The coupling strength between the at least one external antenna 210 and the at least one internal antenna 222 is inversely dependent on the distance (e.g., SFT) between the at least one external antenna 210 and the at least one internal antenna 222.

In certain implementations, upon flowing an electric current through the at least one external antenna 210, the resulting electromagnetic field generates an electric voltage (e.g., induced EMF) in the at least one internal antenna 222 of the implanted device 220 and in the second wire coil 236 of the at least one electromagnetic field detector 230 (e.g., electromagnetic induction). In addition, the electromagnetic fields 234 between the apparatus 200 and the implanted device 220 are affected by the reflected impedance between the at least one external antenna 210 and the at least one internal antenna 222, and these effects are manifested in the detected portions of the electromagnetic fields 234. The voltage induced into the at least one internal antenna 222 by the electromagnetic fields 234 creates an electric current that flows through the load of the at least one internal antenna 222 (e.g., as a primary of an RF transformer). The electromagnetic field created by this electric current opposes the electromagnetic field produced by the at least one external antenna 210, resulting in an increase of the impedance of the at least one external antenna 210 (e.g., reflected impedance).

As can be seen by comparing FIGS. 4A-4D, the detected electromagnetic fields 234 from the apparatus 200 during one or more power transmission phases and/or one or more data transmission phases have amplitudes that are dependent on whether the at least one external antenna 210 is operationally coupled with the at least one internal antenna 222 (e.g., the presence or absence of the implanted device 220) and on the distance between the at least one external antenna 210 and the at least one internal antenna 222 (e.g., the coupling strength between the external and internal antennas 210, 222 is inversely dependent on the distance or SFT). The detected electromagnetic fields 234 have their largest amplitude when the apparatus 200 is not in wireless communication with an implanted device 220 (e.g., the implanted device 220 is not below the apparatus 200, corresponding to FIG. 4A). Furthermore, the amplitudes of the detected electromagnetic fields 234 vary inversely with SFT (e.g., larger amplitudes for small SFT and smaller amplitudes for large SFT) and vary inversely with coupling strength (e.g., larger amplitudes for weak coupling strength and smaller amplitudes for stronger coupling strength). Thus, the amplitudes of the detected electromagnetic fields 234 (e.g., during one or more of the power transmission phases and/or during one or more of the data transmission phases) can be used, at least in part, to generate information indicative of a presence or absence of the implanted device 220 beneath the apparatus 200, indicative of the skin flap thickness, and/or indicative of the coupling strength between the apparatus 200 and the implanted device 220. In certain implementations in which the detector signals 232 are indicative of the amplitudes of the detected electromagnetic fields 234, the circuitry 240 of the apparatus 200 is configured to generate the information indicative of a presence or absence of the implanted device 220 beneath the apparatus 200, indicative of the SFT, and/or indicative of the coupling strength, at least in part in response to the detector signals 232.

In certain implementations, the at least one external antenna 210 has a first tuning frequency and is operationally coupled with the at least one internal antenna 222 having a second tuning frequency different from the first tuning frequency. For example, the at least one external antenna 210 and the at least one internal antenna 222 can be closely tuned to one another (e.g., the first tuning frequency and the second tuning frequency can be within ±20% of one another; within ±10% of one another). The mutual inductance of the external and internal antennas 210, 222 resulting from the coupling between the external and internal antennas 210, 222 (which is inversely dependent on the distance between the external and internal antennas 210, 222) affects the frequency of the electromagnetic fields 234 between the apparatus 200 and the implanted device 220 and the detected portion of these electromagnetic fields 234. The frequency of the detected electromagnetic fields 234 is substantially equal to the first tuning frequency when the apparatus 200 is not in wireless communication with an implanted device 220 (e.g., the implanted device 220 is not below the apparatus 200, so there is no mutual inductance, corresponding to FIG. 4A). In certain implementation in which the detector signals 232 are indicative of the frequency of the detected electromagnetic fields 234 (e.g., during one or more power transmission phases and/or during one or more data transmission phases), the circuitry 240 of the apparatus 200 is configured to generate the information indicative of a presence or absence of the implanted device 220 beneath the apparatus 200 at least in part in response to the detector signals 232. In certain implementations, the at least one electromagnetic field detector 230 (e.g., second wire coil 236) can be automatically tuned to the frequency of the detected electromagnetic fields 234. The frequency of the detected electromagnetic field can be indicative of the tuning frequency of the at least one external antenna 210 and/or the at least one internal antenna 222.

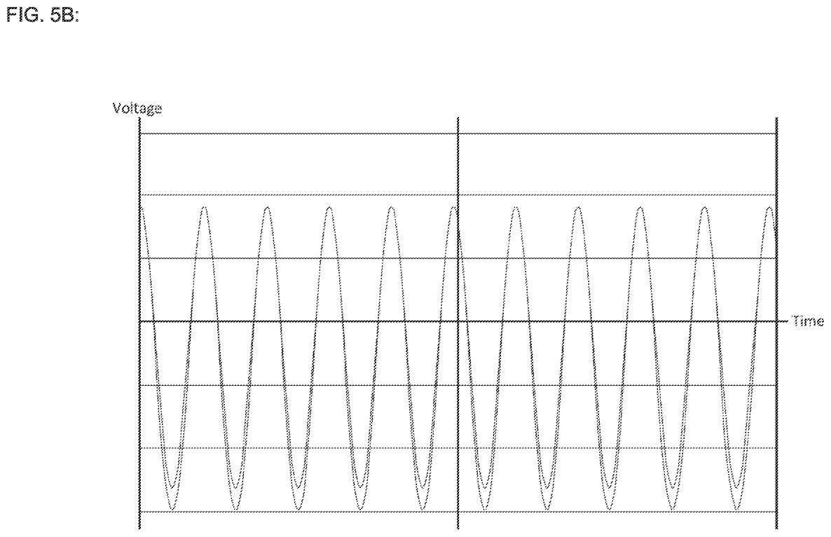

Furthermore, in certain implementations, the frequency of the detected electromagnetic fields 234 varies inversely with the SFT (e.g., farther from the first tuning frequency for small SFT and closer to the first tuning frequency for large SFT) and with the coupling strength (e.g., farther from the first tuning frequency for stronger coupling strengths and closer to the first tuning frequency for weaker coupling strengths). For example, FIGS. 5A and 5B plot the detected electromagnetic fields 234 during a power transmission phase using first wire coil 212 having a tuning frequency of 5 MHz for two different SFT values (e.g., 2 millimeters and 10 millimeters, respectively) in accordance with certain implementations described herein. The detected electromagnetic fields 234 of FIG. 5A have a first frequency (e.g., 5.045 MHz) and the detected electromagnetic fields 234 of FIG. 5B have a second frequency (e.g., 5.004 MHz), the second frequency closer to the tuning frequency of the first wire coil 212 than is the first frequency. In certain implementation in which the detector signals 232 are indicative of the frequency of the detected electromagnetic fields 234 (e.g., during one or more power transmission phases and/or during one or more data transmission phases), the circuitry 240 of the apparatus 200 is configured to generate the information indicative of the SFT and/or indicative of the coupling strength between the apparatus 200 and the implanted device 220, at least in part in response to the detector signals 232.

In certain implementations, upon the information indicating that the apparatus 200 is not operationally coupled with an implanted device 220, the circuitry 240 can cease attempting to wirelessly transfer power to the implanted device 220, thereby saving battery power that would otherwise be wasted by continuing to attempt to establish communication (e.g., by flowing electrical current through the first wire coil 212). In certain implementations, upon the information indicating the coupling strength between the apparatus 200 and the implanted device 220, the circuitry 240 can automatically adjust an amplitude of the electrical power and/or data being wirelessly transmitted from the apparatus 200 to the implanted device 220. For example, in response at least in part to the information, the circuitry 240 can generate and transmit control signals 242 to the at least one antenna driver 214 to increase the amplitude of the electrical power and/or data being wirelessly transmitted from the apparatus 200 when the coupling strength is relatively weak (e.g., such that the amplitude of the electrical power and/or data received by the implanted device 220 satisfies an operationally acceptable threshold level), thereby automatically maintaining functional wireless communication.

FIG. 4E shows the detected electromagnetic fields 234 during power transmission phases, data transmission phases, and quiet phases while the apparatus 200 and the implanted device 220 are exposed to externally generated electromagnetic fields from other electromagnetic field sources. These externally generated electromagnetic fields can cause external interference of the wireless transmission of power and/or data between the apparatus 200 and the implanted device 220. These externally generated electromagnetic fields can be measured (e.g., amplitude; frequency) during one or more power transmission phases, one or more data transmission phases, and/or one or more quiet phases. For example, as shown in FIG. 4E, although the at least one external antenna 210 receives a power transmission electric current having a substantially constant magnitude (e.g., variations less than ±10%, less than ±5%) from the at least one antenna driver 214 during the power transmission phases (see, e.g., FIGS. 4A-4D), due to the external interference, the electromagnetic fields 234 detected during the power transmission phases has a varying magnitude (e.g., variations greater than ±10%, greater than ±5%). Similarly, although the at least one external antenna 210 receives a data transmission electric current having a substantially constant magnitude (e.g., variations less than ±10%, less than ±5%) from the at least one antenna driver 214 during the data transmission phases, due to the external interference, the electromagnetic fields 234 detected during the data transmission phases has a varying magnitude (e.g., variations greater than ±10%, greater than ±5%).

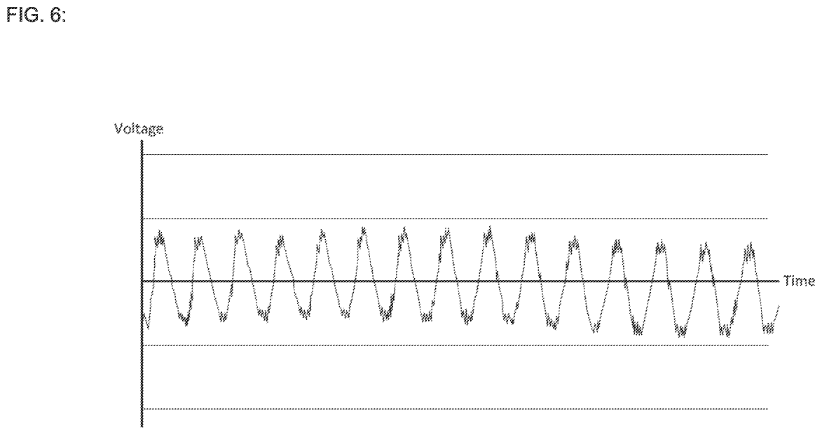

In certain implementations, the presence of external interference can cause a change of frequency of the electromagnetic fields 234 detected during either the power transmission phases and/or the data transmission phases, as compared to the frequency of the electromagnetic fields 234 during an absence of external interference. In addition, during the quiet phases in which the at least one external antenna 210 receives substantially zero electric current, the electromagnetic fields 234 detected during the quiet phases has a substantially non-zero amplitude. For example, FIG. 6 plots the electric voltage 237 corresponding to electromagnetic fields 234 detected during a quiet phase in accordance with certain implementations described herein. The electric voltage 237 of FIG. 6 indicates that the apparatus 200 is exposed to an electromagnetic field generated by an external source.

In certain implementations, the information indicative of the presence or absence of external interference is generated in response, at least in part, to the detected electromagnetic fields 234 during one or more quiet phases (e.g., while neither electrical power nor data is wirelessly transmitted between the apparatus 200 and the implanted device 220). For example, by measuring the electric voltage 237 generated by the second wire coil 236 during one or more quiet phases, the external interference field and its parameters (e.g., frequency; amplitude) can be determined. In certain other implementations, the information indicative of the presence or absence of external interference is generated in response, at least in part, to the detected electromagnetic fields 234 during one or more power transmission phases (e.g., while electrical power is wirelessly transmitted between the apparatus 200 and the implanted device 220) and/or during one or more data transmission phases (e.g., while data is wirelessly transmitted between the apparatus 200 and the implanted device 220).

In certain implementations, the implanted device 220 is configured to generate and transmit telemetry data signals (e.g., data pulses) back to the apparatus 200. However, under some conditions, the apparatus 200 may not receive or recognize these telemetry data signals. Reasons for failure to receive or recognize the telemetry data signals include but are not limited to: absence of the implanted device 220; lack of sufficient coupling between the apparatus 200 and the implanted device 220 (e.g., due to large SFT); interference from an externally generated electromagnetic field; incorrect operation of the implanted device 220; incorrect threshold levels of the apparatus 200. In certain implementations, the method 300 can further comprise restoring telemetry data from telemetry data signals affected by external interference. For example, the apparatus 200 can subtract the electromagnetic fields 234 detected (e.g., measured and stored) during a first time slot in which neither electrical power nor data is wirelessly transmitted between the apparatus 200 and the implanted device 220 (e.g., during a portion of a quiet phase in which telemetry data signals are also not being transmitted) from the telemetry data signals received by the apparatus 200 from the implanted device 220 during a second time slot (e.g., another portion of a quiet phase) in which telemetry data signals are being transmitted. During the first time slot (e.g., between the last telemetry pulse and the beginning of a power transmission phase), the detected electromagnetic fields 234 correspond only to the externally generated electromagnetic interference and during the second time slot, the detected electromagnetic fields 234 correspond to a mixture of the telemetry data signals and the externally generated electromagnetic interference. In this way, the externally generated electromagnetic interference can be removed, thereby extracting or restoring the telemetry data signals.

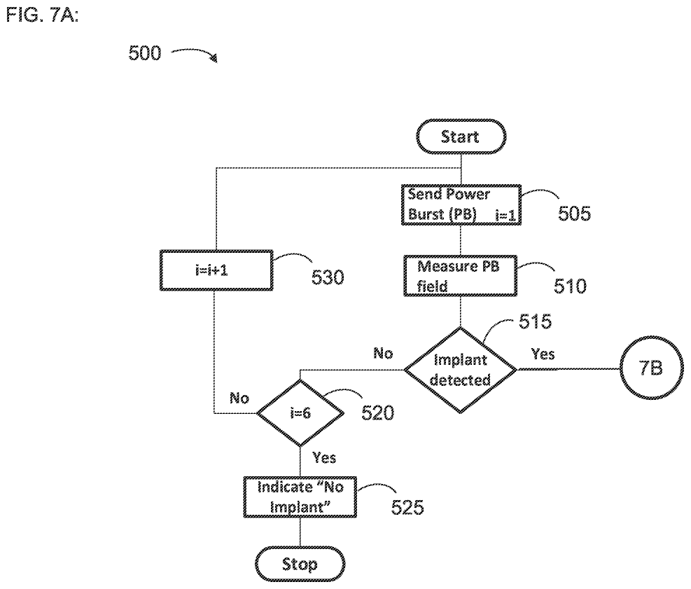

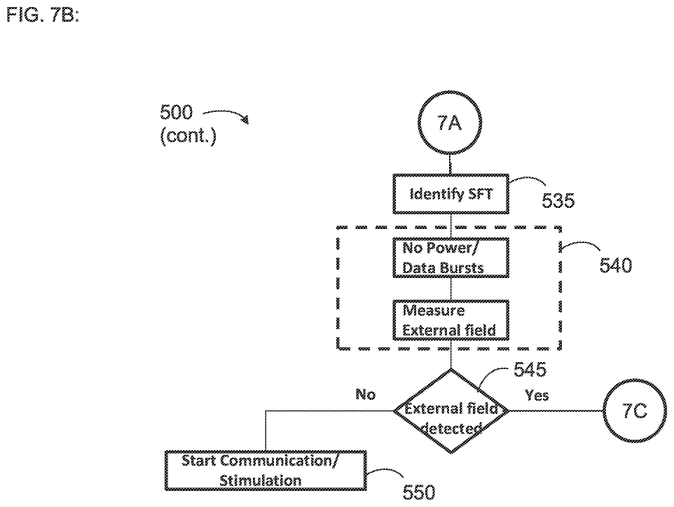

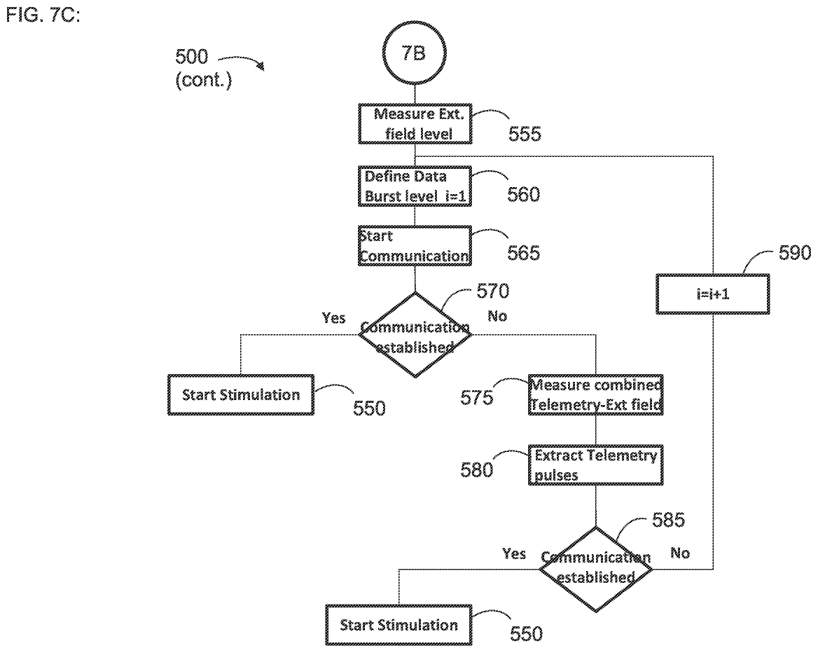

FIGS. 7A-7C are a flow diagram of an example method 500 of detection of an internal device and diagnosis of the RF communication link between an external device (e.g., apparatus 200) and the internal device (e.g., implanted device 220) in accordance with certain implementations described herein. While the method 500 is described by referring to some of the structures of the example apparatus 200 of FIGS. 1A-1D and 2A-2B, other apparatus and systems with other configurations of components can also be used to perform the method 500 in accordance with certain implementations described herein. For example, the method 500 can be performed by an external portion of a medical device or system (e.g., an auditory or visual prosthesis system; cardiac pacemaker or defibrillator system) configured to have a magnetic induction link configured to transmit power and/or data to an internal portion (e.g., implanted component) of the medical device or system. For example, evaluation and detection operational blocks of the method 500 can be performed by the circuitry 240 and the measurement operational blocks of the method 500 can be performed using the at least one electromagnetic field detector 230 and the circuitry 240.

FIG. 7A shows the flow diagram for a portion of the method 500 which detects the presence or absence of the internal device. In an operational block 505, the method 500 comprises wirelessly transmitting a power burst (PB) from the external device, the PB configured to provide power to an internal device (e.g., a power transmission phase of the external device). In the operational block 505, the method 500 also initializes a first counter to equal one (e.g., i=1), the first counter tracking the number of attempts to detect the presence of the internal device. In an operational block 510, the method 500 further comprises measuring the electromagnetic field of the PB (e.g., using at least one electromagnetic field detector 230 to measure an amplitude and/or a frequency of the PB electromagnetic field 234). In an operational block 515, the method 500 further comprises determining whether the internal device is detected. For example, the circuitry 240 of the apparatus 200 can evaluate whether the measured amplitude and/or frequency of the PB electromagnetic field is indicative of the presence or the absence of an internal device. If the circuitry 240 does not determine that the internal device is detected (e.g., “No” branch from operational block 515), the method 500 proceeds to operational block 520 which evaluates whether the first counter is equal to a predetermined number of attempts to detect the presence of the internal device (e.g., number of attempts equal to six; i=6). If the circuitry 240 determines that the predetermined number of attempts have been made (e.g., “Yes” branch from operational block 520), the method 500 proceeds to operational block 525 which generates a signal indicative of the absence of an internal device (e.g., “No Implant” signal provided to the recipient and/or a practitioner), and the method 500 automatically ceases further power transmission. If the circuitry 240 determines that the predetermined number of attempts have not yet been made (e.g., “No” branch from operational block 520), the method 500 proceeds to operational block 530 which increments the first counter (e.g., i=i+1) and the method 500 repeats operational blocks 505, 510, and 515. If the circuitry 240 determines that the internal device is detected (e.g., “Yes” branch from operational block 515), the method 500 proceeds to the operational blocks shown in FIG. 7B.

FIG. 7B shows the flow diagram for a portion of the method 500 which identifies the skin flap thickness (SFT) and detects the presence or absence of externally generated electromagnetic fields that can degrade the RF communication link between the external device and the internal device. In an operational block 535, the method 500 further comprises identifying the SFT value for the recipient (e.g., using the amplitude and/or frequency of the PB field measured in the operational block 510). For example, the SFT can be identified by comparing the measured value of the amplitude and/or frequency of the PB field with predetermined values (e.g., in a table) corresponding to predetermined SFT values (e.g., interpolating from the measured value using the predetermined values of the amplitude and/or frequency and the corresponding predetermined SFT values to calculate a measured SFT value). The table of amplitude and/or frequency values with the corresponding SFT values can be stored in and accessed from a non-transitory memory device (e.g., random-access memory integrated circuit; flash memory; other tangible data storage device) by the processor circuitry 244. Depending on the recipient's SFT, the method 500 can further comprise setting the power and/or data transmission amplitudes from the external device (e.g., the electrical current flowing through the at least one external antenna 210) to sufficient value to achieve the power and/or data transmission across the SFT of the recipient. In certain such implementations, said setting the power and/or data transmission amplitudes at a proper value for the recipient's SFT can save battery power that would otherwise be wasted by flowing excessively high electric currents through the at least one external antenna 210.

In an operational block 540, the method 500 further comprises measuring the electromagnetic fields during a time period in which neither power nor data are being transmitted by the external device (e.g., during a portion of the quiet phase of the external device) and telemetry signals are not being transmitted by the internal device. In an operational block 545, the method 500 further comprises determining whether an externally generated electromagnetic field is detected. For example, the circuitry 240 of the apparatus 200 can evaluate whether the measured amplitude of the measured electromagnetic field is sufficient to degrade the RF communication link. If the circuitry 240 determines that the measured amplitude is insufficient to cause substantial degradation of the RF communication link (e.g., “No” branch from operational block 545), the method 500 proceeds to operational block 550 in which the external device and the internal device begin operation (e.g., begin wireless communication and/or stimulation for operation of the auditory prosthesis system). If the circuitry 240 determines that the measured amplitude is sufficient to cause substantial degradation of the RF communication link (e.g., “Yes” branch from operational block 545), the method 500 proceeds to the operational blocks shown in FIG. 7C.

FIG. 7C shows the flow diagram for a portion of the method 500 which extracts information from telemetry signals received by the external device from the internal device, which can include restoring telemetry signals corrupted by the externally generated electromagnetic fields. In an operational block 555, the method 500 further comprises measuring and recording the externally generated electromagnetic fields during a time period in which neither power nor data are being transmitted by the external device (e.g., during a portion of the quiet phase of the external device) and telemetry signals are not being transmitted by the internal device. This measurement can be part of the measurement of the operational block 540 or can be a separate measurement. In an operational block 560, the method 500 further comprises setting the amplitude of the telemetry data bursts from the internal device and initializing a second counter to equal one (e.g., i=1), the second counter tracking the number of attempts to transmit telemetry data from the internal device to the external device. In an operational block 565, the method 500 further comprises starting communication of telemetry data (e.g., starting to transmit telemetry signals from the internal device; starting to receive telemetry signals by the external device). In an operational block 570, the method 500 further comprises determining whether telemetry data is able to be extracted from the telemetry signals received by the external device. For example, the circuitry 240 of the apparatus 200 can evaluate whether the received telemetry signals are sufficiently uncorrupted for the circuitry 240 to extract the telemetry data from the received telemetry signals. If the circuitry 240 determines that the received telemetry signals are sufficiently uncorrupted (e.g., “Yes” branch from operational block 570), the method 500 proceeds to the operational block 550 and the external device and the internal device begin operation (e.g., begin wireless communication and/or stimulation for operation of the auditory prosthesis system) which includes extracting the telemetry data from the received telemetry signals.

If the circuitry 240 determines that the received telemetry signals are corrupted (e.g., “No” branch from operation block 570), the method 500 proceeds to restore the telemetry signals so as to be able to extract the telemetry data. In an operational block 575, the method 500 further comprises measuring and recording the electromagnetic fields during a time period in which neither power nor data are being transmitted by the external device (e.g., during a portion of the quiet phase of the external device) and telemetry signals are being transmitted by the internal device. The measured and recorded electromagnetic fields represent a combination of the externally generated electromagnetic fields and the telemetry signals from the internal device. In an operational block 580, the method 500 further comprises extracting the telemetry data (e.g., telemetry pulses) from the combination. For example, the measured and recorded externally generated electromagnetic fields from the operational block 555 (e.g., during a time period in which neither power nor data are being transmitted by the external device and telemetry signals are not being transmitted by the internal device) can be subtracted from the measured and recorded electromagnetic fields from the operational block 575, thereby leaving only the contribution from the telemetry signals. In an operational block 585, the method 500 further comprises determining whether the telemetry data is able to be extracted from the contribution from the telemetry signals of operational block 580. If the circuitry 240 determines that the telemetry data can be extracted from the contribution from the telemetry signals of operational block 580 (e.g., “Yes” branch from operational block 585), the method 500 proceeds to the operational block 550 and the external device and the internal device begin operation (e.g., begin wireless communication and/or stimulation for operation of the auditory prosthesis system) which includes extracting the telemetry data from the contribution from the telemetry signals of operational block 580. If the circuitry 240 determines that the telemetry data cannot be extracted from the contribution from the telemetry signals of operational block 580 (e.g., “No” branch from operational block 585), the method 500 proceeds to operational block 590 which increases the amplitude of the telemetry data bursts from the internal device and increments the second counter (e.g., i=i+1) and the method 500 repeats operational blocks 560, 565, and 570.

In certain implementations, the operational blocks of FIG. 7C can further comprise adjusting the amplitude of the data transmission signals from the external device to ensure proper data transfer during the data transmission phases. For example, in a manner similar to that described herein with regard to the telemetry signals, the method 500 can comprise setting an initial value of the data transmission signals (e.g., initial value of the electric current flowing through the at least one external antenna 210 during the data transmission phases), determining whether there is sufficient communication of the data transmission signals via the RF communication link, and if there is not, subtracting the externally generated electromagnetic fields from the combination of the externally generated electromagnetic fields and the data transmission signals from the external device.

In certain implementations, the at least one electromagnetic field detector 230 is used in a closed loop system configuration. For example, the at least one electromagnetic field detector 230 can measure the magnitude of the electromagnetic field during one or more power transmission phases and can send a feedback signal and/or information to the circuitry 240 (e.g., sound processor controller). Based on the feedback signal and/or information regarding the measured electromagnetic field magnitude, the circuitry 240 can: identify whether the detected electromagnetic fields 234 are created by the at least one external antenna 210 only (e.g., there is no implanted device coupled to the external device), identify whether the detected electromagnetic fields 234 are created by the at least one external antenna 210 and the at least one internal antenna 222 (e.g., combined electromagnetic fields), and/or determine a distance between the at least one external antenna 210 and the at least one internal antenna 222 (e.g., SFT). In certain implementations, the amplitude of the electrical power and/or data being wirelessly transmitted from the apparatus 200 to the implanted device 220 can be adjusted using a closed loop system in which both the forward (e.g., data) and backward (e.g., telemetry) RF communication link are functioning. For example, in certain implementations in which the data signals are corrupted due to presence of an externally generated electromagnetic field that interferes with the data signals, the circuitry 240 can increase the data signal amplitude. For another example, in certain implementations in which the data signals are corrupted due to ringing due to a small SFT value, the circuitry 240 can decrease the data signal amplitude. For another example, in certain implementations in which the data signals are corrupted by both externally generated electromagnetic fields and ringing, the circuitry 240 can adjust both the power signal amplitude and the data signal amplitude (e.g., to adapt the levels for optimum performance and in minimum time).

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having an external portion of the implantable medical device wirelessly receive information from an implanted portion of the implantable medical device while the external portion wirelessly transmits power to the implanted portion.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
at least one external antenna external to a recipient's body and configured to be in wireless communication with at least one internal antenna of an implanted device within the recipient's body;
at least one electromagnetic field detector external to the recipient's body and configured to generate detector signals in response to electromagnetic fields generated by the at least one external antenna, by the at least one internal antenna, and/or by other electromagnetic field sources; and
circuitry in operable communication with the at least one external antenna and the at least one electromagnetic field detector, the circuitry configured to receive the detector signals from the at least one electromagnetic field detector and, in response at least in part to the detector signals, transmit control signals to the at least one external antenna.

2. The apparatus of claim 1, wherein the at least one external antenna comprises a first wire coil and at least one antenna driver in operative communication with the first wire coil.

3. The apparatus of claim 1, wherein the at least one electromagnetic field detector comprising a second wire coil and a detector interface in operative communication with the second wire coil.

4. The apparatus of claim 3, wherein the second wire coil is between the at least one external antenna and the recipient's body.

5. The apparatus of claim 3, wherein the detector interface comprises a differential amplifier having sufficiently large impedance that an electrical current does not flow within the second wire coil in response to the electromagnetic fields.

6. The apparatus of claim 1, further comprising a housing containing the at least one external antenna and the at least one electromagnetic field detector.

7. The apparatus of claim 1, wherein the circuitry comprises processor circuitry configured to generate the control signals in response to the detector signals.

8. The apparatus of claim 1, wherein the apparatus comprises an external portion of an acoustic prosthesis system, the implanted device comprises an implanted portion of the acoustic prosthesis system.

9. The apparatus of claim 8, wherein the circuitry comprises digital sound processing circuitry configured to receive data signals indicative of sound detected by at least one microphone of the acoustic prosthesis system, and the circuit configured to generate the control signals in response to at least the data signals.

10. The apparatus of claim 1, wherein the at least one electromagnetic field detector is configured to generate the detector signals in response to the electromagnetic fields generated by the at least one external antenna, and in response to the detector signals, the circuitry is configured to evaluate operation of the apparatus.

11. A method comprising:
wirelessly transmitting electrical power and/or data from an external portion of a medical implant system outside a recipient's body, the external portion configured to be in wireless communication with an internal portion of the medical implant system within the recipient's body;
detecting electromagnetic fields generated by the external portion, by the internal portion, and/or by other electromagnetic field sources; and
generating information in response to the detected electromagnetic fields, the information indicative of at least one of:
a presence or absence of the internal portion;
a distance between the external portion and the internal portion;
a presence or absence of external interference of wireless communication between the external portion and the internal portion;
an operational state of the external portion of the medical implant system; and
a coupling strength between the external portion and the internal portion.

12. The method of claim 11, wherein the information is generated in response, at least in part, to the detected electromagnetic fields while wirelessly transmitting electrical power from the external portion.

13. The method of claim 11, wherein the information indicative of the presence or absence of external interference is generated in response, at least in part, to the detected electromagnetic fields while neither electrical power nor data is wirelessly transmitted between the external portion and the internal portion.

14. The method of claim 11, wherein said detecting comprises detecting the electromagnetic fields generated by the external portion while the external portion is not in operable communication with the internal portion, and said generating comprises generating the information indicative of the operational state of the external portion.

15. The method of claim 11, wherein the information indicative of a coupling strength between the external portion and the internal portion is generated in response, at least in part, to the detected electromagnetic fields while wirelessly transmitting data from the external portion to the internal portion.

16. The method of claim 11, wherein the information indicative of the distance between the external portion and the internal portion is generated by comparing an amplitude and/or frequency value of the detected electromagnetic field with predetermined amplitude and/or frequency values corresponding to predetermined distances.

17. The method of claim 11, further comprising receiving telemetry data signals transmitted from the internal portion, determining whether the telemetry data signals are corrupted by external interference, and restoring telemetry data from the telemetry data signals corrupted by the external interference by subtracting the electromagnetic fields detected while neither electrical power nor data is wirelessly transmitted between the external portion and the internal portion from the telemetry data signals received by the external portion from the internal portion.

18. The method of claim 11, further comprising responding to the information by adjusting an amplitude of the electrical power and/or the data wirelessly transmitted from the external portion to the internal portion.

19. The method of claim 11, wherein the medical implant system comprises an auditory prosthesis system.

20. A method comprising:
wirelessly transmitting electrical power and/or data from an external portion of a medical implant system outside a recipient's body;
detecting electromagnetic fields generated by the external portion;
generating information in response to the detected electromagnetic fields, the information indicative of a presence or absence of an internal portion of the medical implant system implanted within the recipient's body; and
in response to the information being indicative of an absence of the internal portion, terminating said wirelessly transmitting electrical power and/or data from the external portion.

21. The method of claim 20, further comprising, in response to the information being indicative of an absence of the internal portion, generating a signal indicative of the absence of the internal portion, the signal configured to be received by a user of the medical implant system.

22. The method of claim 20, wherein the information is generated in response, at least in part, to the detected electromagnetic fields while wirelessly transmitting electrical power from the external portion.

23. The method of claim 20, wherein the information indicative of an absence of an internal portion is generated after a predetermined number of attempts to detect the presence of the internal portion.

24. A method comprising:
wirelessly transmitting electrical power and/or data from an external portion of a medical implant system outside a recipient's body;
detecting electromagnetic fields generated by the external portion;
generating information in response to the detected electromagnetic fields, the information indicative of an operational state of the external portion; and
in response to the information, modifying said wirelessly transmitting electrical power and/or data from the external portion.

25. The method of claim 24, further comprising, in response to the information being indicative of the operational state, generating a signal indicative of the operational state of the external portion, the signal configured to be received by a user of the medical implant system.

26. The method of claim 24, wherein the information is generated in response, at least in part, to the detected electromagnetic fields while the external portion is not in operational communication with an internal portion of the medical implant system implanted within the recipient's body.

27. The method of claim 26, wherein said detecting the electromagnetic fields comprises measuring a duration and amplitude of RF fields generated by the external portion during a power transmission operational phase of the external portion.
